(19) United States
(12) Patent Application Publication
(10) Pub. No.: US 2011/0021824 A1
(43) Pub. Date: Jan. 27, 2011

(54) ADSORPTIVE PURIFICATION METHOD FOR IODIXANOL

(75) Inventors: LARS TERJE HOLMAAS, SPANGEREID (NO); OLE MAGNE HOMESTAD, SPANGEREID (NO); DIDRIK MALTHE-SORENSSEN, SPANGEREID (NO); ERIC PRESSMAN, EAST GREENBUSH, NY (US); ALBERT SANTO STELLA, VOORHEESVILLE, NY (US)

Correspondence Address: GE HEALTHCARE, INC., IP DEPARTMENT 101 CARNEGIE CENTER, PRINCETON, NJ, 08540-6231, US

(73) Assignee: GE HEALTHCARE AS, OSLO (NO)

(21) Appl. No.: 12/581,957

(22) Filed: Oct. 20, 2009

Related U.S. Application Data
(60) Provisional application No. 61/227,103, filed on Jul. 21, 2009.

(51) Int. Cl. C07C 227/40

(52) U.S. Cl. 564/153

(57) ABSTRACT

This invention relates generally to non-ionic X-ray contrast agents. It further relates to a method of using solid adsorbents to reduce backpeaks content in the purification of iodixanol. In particular, it relates to the use of non-polar organic adsorbents with the average pore diameter smaller than about 30 nm. Specific examples of adsorbents of the instant invention include non-polar acrylic ester, di-vinyl benzene resins, poly-styrene di-vinyl benzene resins, and carbon adsorbents. In certain embodiments, upwards of 30% of the backpeak levels and 60% N-acetyl cyclic iodixanol levels are reduced for a 5% loss of iodixanol.

### CROSS REFERENCE TO RELATED APPLICATIONS

The present application claims benefit of priority under 35 U.S.C. §119(e) to U.S. Provisional Application No. 61/227,103 filed Jul. 21, 2009, the entire disclosure of which is hereby incorporated by reference.

### TECHNICAL FIELD

This invention relates generally to non-ionic X-ray contrast agents. It further relates to a method of using solid adsorbents to reduce backpeaks content in the purification of iodixanol. In particular, it relates to the use of non-polar organic adsorbents with the average pore diameter smaller than about 30 nm.

### BACKGROUND OF THE INVENTION

Iodixanol is a non-ionic X-ray contrasting agent produced in large quantities by GE Healthcare in Lindesnes, Norway. The industrial production of iodixanol involves a multistep chemical synthesis. The lowest yield is in the final step, involving coupling of the intermediate 5-acetamido-N,N′-bis(2,3-dihydroxypropyl)-2,4,6-triiodoisophthalamide (“Compound A”) with epichlorohydrin under basic conditions. Yield losses are attributed to side reactions leading to byproducts (“backpeaks”). These backpeaks must be minimized to provide iodixanol that meets regulatory requirements like those mandated by US Pharmacopeia.

While the purification of iodixanol is necessary and extensive, it is also important that such process does not lead to substantial loss of iodixanol. There exists a need for economic procedures to achieve the desired purity of iodixanol, while minimizing the loss of the final product during the purification process.

### BRIEF DESCRIPTION OF THE DRAWINGS

FIG. 1 illustrates iodixanol purification in aqueous solution with various mostly non-polar adsorbents.

FIG. 2 illustrates aqueous iodixanol solution backpeak reduction from control for best non-polar adsorbents.

FIG. 3 illustrates aqueous iodixanol solution N-acetyl cyclic iodixanol reduction using mostly non-polar adsorbents.

FIG. 4 illustrates backpeak adsorption isotherms from aqueous solution for CG71M resin and BGHHM and NC01-125 carbons.

FIG. 5 illustrates iodixanol adsorption isotherms from aqueous solution for CG71M resin and BGHHM and NC01-125 carbons

FIG. 6 illustrates average performance of adsorbents for purifying iodixanol in a 20.8 wt % aqueous solution, showing the percentage of the original mass adsorbed by the adsorbent.

FIG. 7 illustrates average separation factors for iodixanol to backpeaks and iodixanol to N-acetyl cyclic iodixanol in aqueous solutions at 25° C.

FIG. 8 illustrates cumulative pore volume as a function of average pore size for BGHHM Carbon.

### SUMMARY OF THE INVENTION

The present invention provides a large scale purification process of iodixanol. This process involves the use of non-polar organic adsorbents in the treatment of bulk iodixanol, where the average pore diameter is smaller than about 30 nm. Specific examples of adsorbents of the instant invention include non-polar acrylic ester, di-vinyl benzene resins, poly-styrene di-vinyl benzene resins, and carbon adsorbents. In some preferred embodiments, adsorbents with 90% of the pore volume smaller than 30 nm are employed. In other embodiments, adsorbents with surface area for adsorption about 1350 m²/g of adsorbent are used.

The less polar impurities include N-acetyl cyclized iodixanol, cyclized iodixanol, and primary o-alkyl trimer. In certain embodiments, upwards of 30% of the backpeak levels and 60% N-acetyl cyclic iodixanol levels are reduced for a 5% loss of iodixanol. For example, upwards of 30% of the backpeak levels reductions may be obtained for a 2:1 solids to adsorbent mass ratio. In another example, separation factors of four may be obtained between iodixanol and backpeaks in adsorptive purification, with about a 20% reduction in backpeaks for a 5% loss of iodixanol to the adsorbed phase.

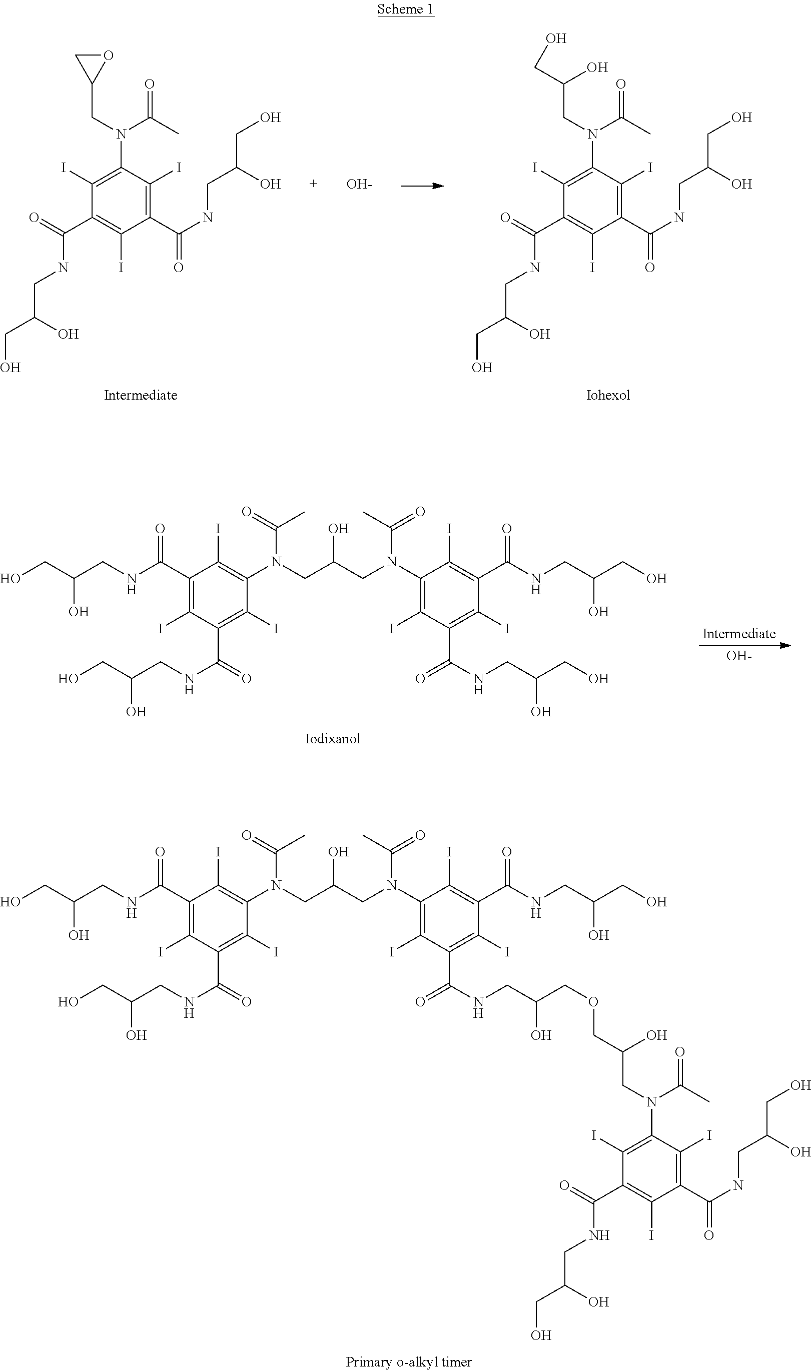

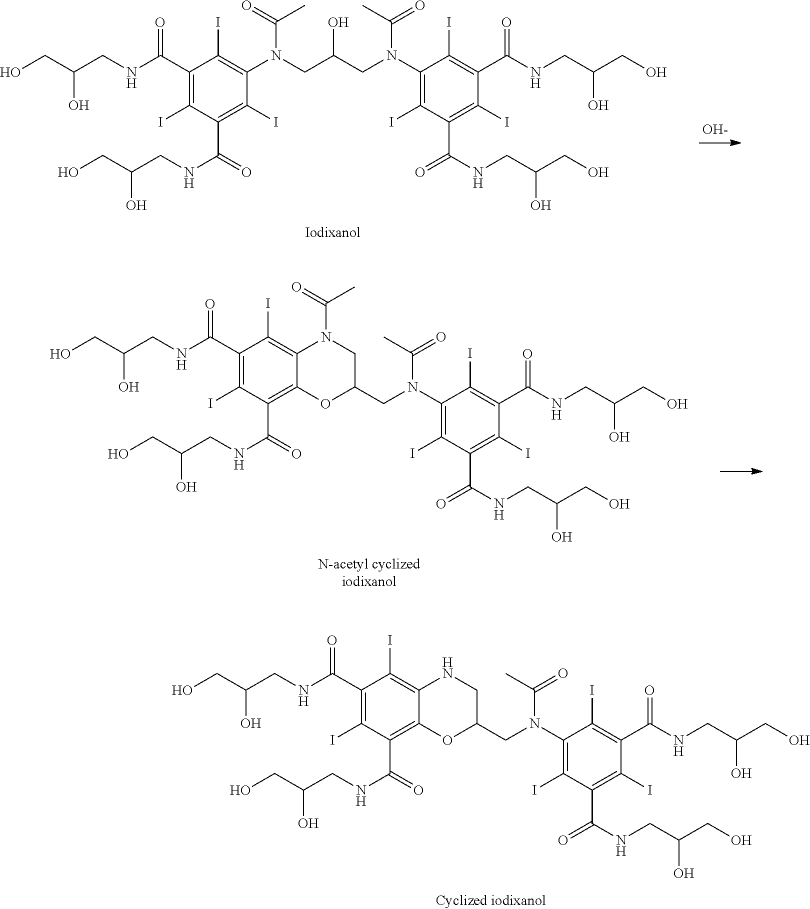

### DETAILED DESCRIPTION OF THE INVENTION

The instant invention is directed to purifying bulk iodixanol using non-polar organic adsorbents, where the average pore diameter is smaller than about 30 nm. In a preferred embodiment, analyses on iodixanol solutions before and after treatment with the adsorbents are performed by High Performance Liquid Chromatography (HPLC). For example, based on a HPLC assay, backpeaks as the major contaminants may be at 0.80-0.90 wt %.

In some embodiments, water is chosen as the solvent for iodixanol in order to exploit the more non-polar nature of the backpeaks relative to iodixanol. This is to create a very polar interaction of iodixanol with water, and a more hydrophobic environment on the adsorbent, to selectively remove backpeaks. This strategy is consistent with the results of reversed phase HPLC conditions, where backpeaks are selectively adsorbed to a greater extent (i.e. have longer retention times than iodixanol) onto a non-polar C18 column when eluting a primarily aqueous mobile phase.

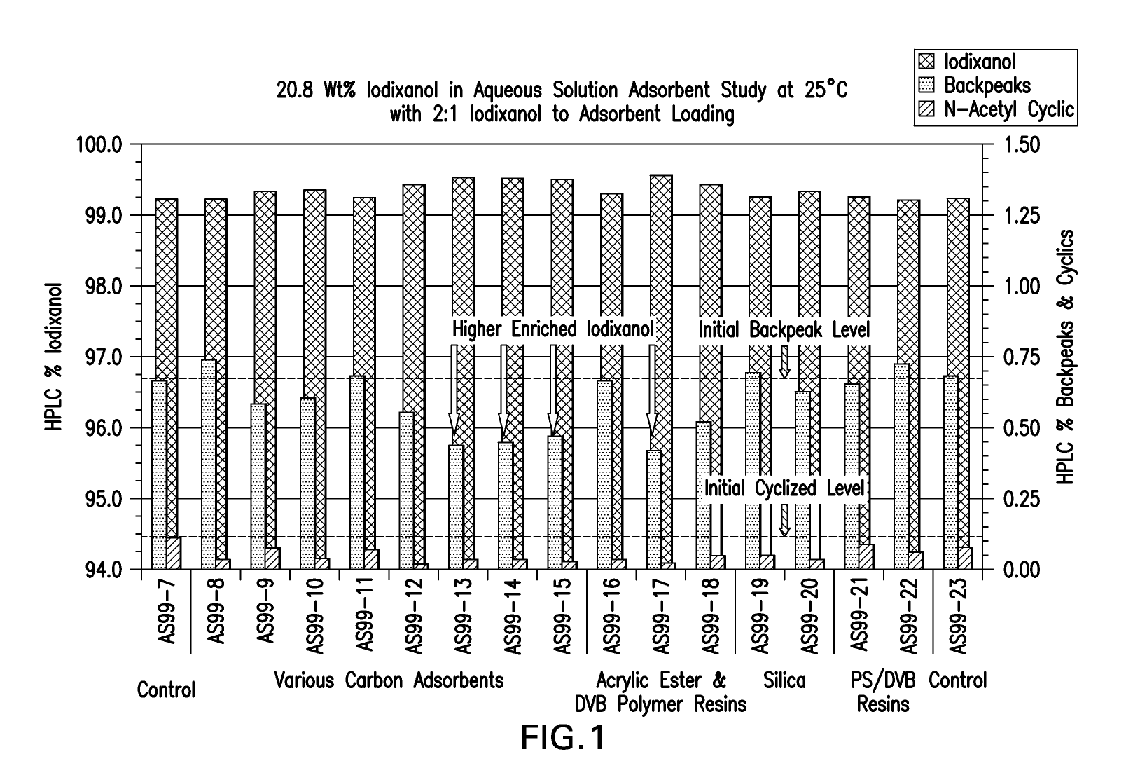

Among the preferred adsorbents selected were the non-polar acrylic ester and di-vinyl benzene resins, as well as a set of poly-styrene di-vinyl benzene resins, and a number of carbon adsorbents. For contrast, a set of polar silica adsorbents was also studied. A 20.8 wt % solids aqueous iodixanol solution was contacted with the adsorbents at 25° C. in a mass ratio of 2:1 iodixanol to adsorbent in order to see if there was a dramatic effect on iodixanol purity with the high loading of adsorbent (FIG. 1).

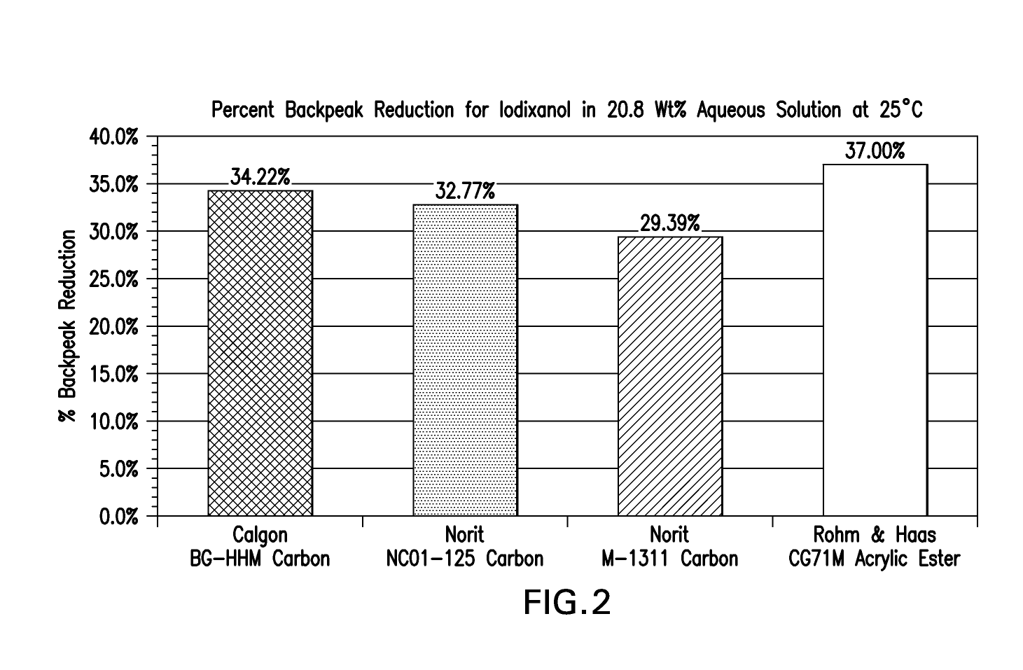

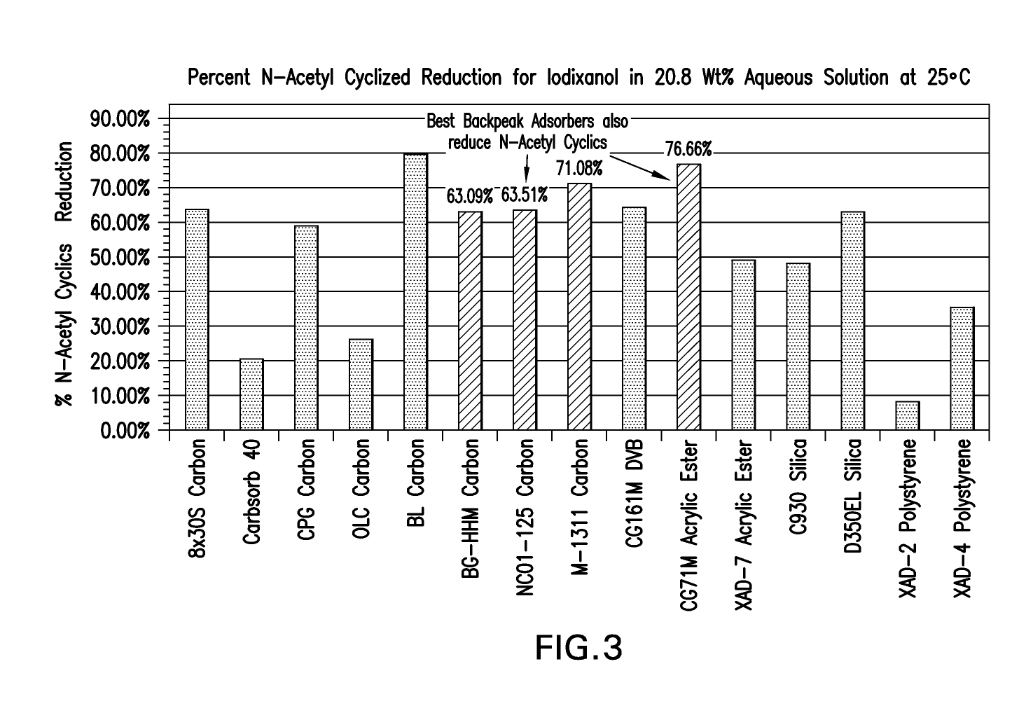

The data show that certain types of carbon and acrylic ester resin adsorbents purify the iodixanol from the less polar backpeaks and N-acetyl cyclic iodixanol in aqueous solution. The amount of backpeak reduction is shown in FIG. 2 for the three carbons and the acrylic ester resin that performed the best in purifying the iodixanol. These four adsorbents reduced the backpeak level by 30% or more and are all commercially available. In addition, cyclic iodixanol is also reduced by 65-70% as shown in FIG. 3.

Although the CG71M has a lower surface area (500 m²/g) than BGHHM Carbon (1350 m²/g), one important comparison for adsorbents is capacity for the desired material to adsorb relative to the product. The preferred way to test this is to perform isotherm studies. To measure adsorption isotherms, known weights of dry adsorbent were added to aliquots of the standard aqueous iodixanol solution of 20.8 wt % solids in mass ratios of 4:1 up to 30:1 iodixanol to adsorbent. The slurries were contacted with mixing for 24 hours, and then aliquots of the resulting supernatant liquid were analyzed for iodixanol, backpeak and N-acetyl cyclic iodixanol content by the standard HPLC analysis.

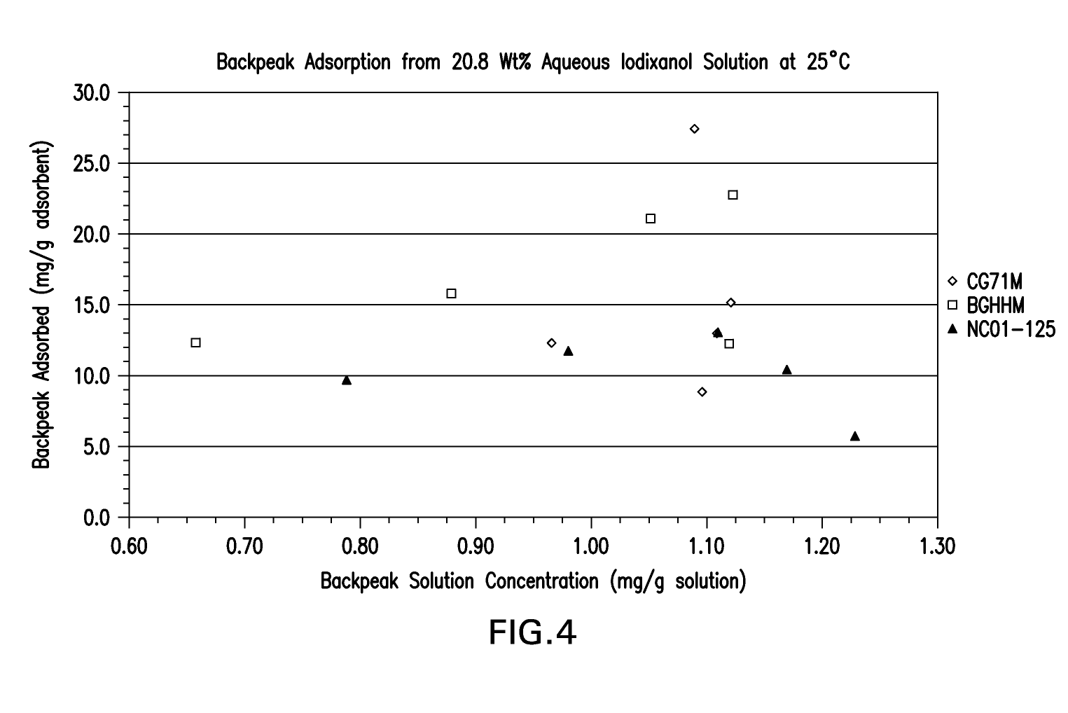

The results for backpeak adsorption as a function of concentration are shown in FIG. 4. The data are plotted as amount of backpeak adsorbed per mass of adsorbent versus the mass concentration of backpeak in the solution at equilibrium.

The scatter in FIG. 4 is due to several factors. The measurements are done by dilution of 200 micro-liters of the supernatant liquid with water and then injection into the HPLC. Although the masses are recorded to four decimal places, there are still potential inaccuracies associated with taking a small sample of the solution. In addition, to get a measured mass concentration from the HPLC, the area for the peak from the HPLC UV measurement at 254 nm is correlated for the control samples, knowing the concentration of each component in the control. The area response of the adsorbed samples is then taken as a ratio of it to the control in order to determine the mass concentration of the sample. In addition, there could be some imprecision involved in the manual integration of the peaks. Despite the scatter, the data show that BGHHM has a better capacity for backpeaks than NC01-125.

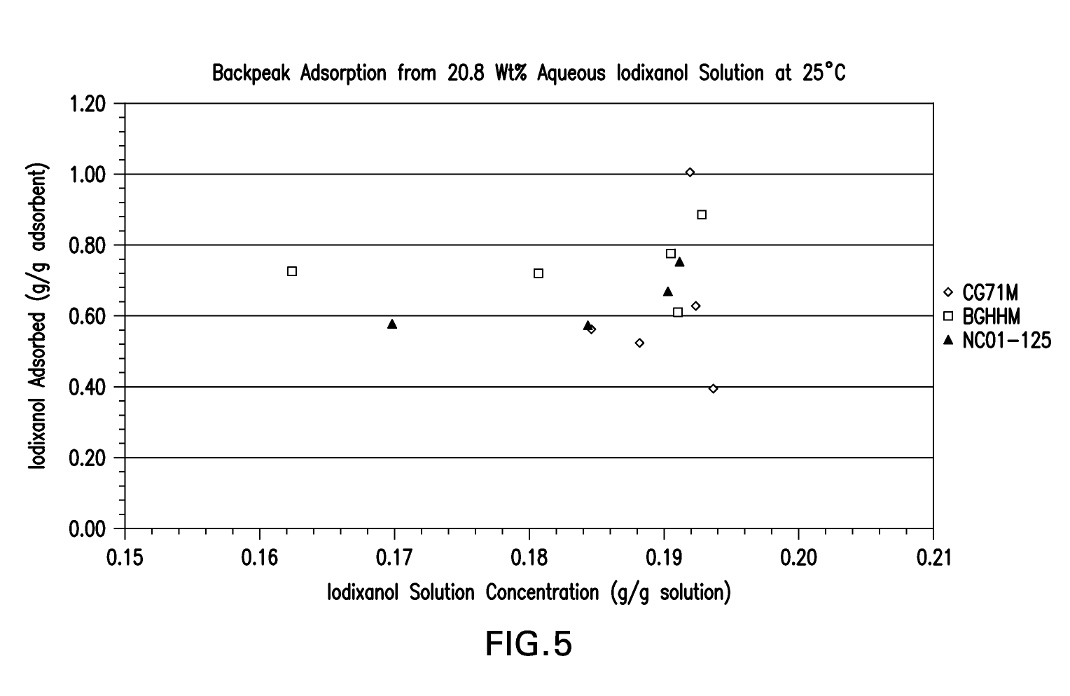

Similar results are shown for iodixanol adsorption as a function of loading. FIG. 5 shows that all adsorbents adsorb only fairly small amounts of iodixanol relative to the solution concentration of nearly 20-wt %.

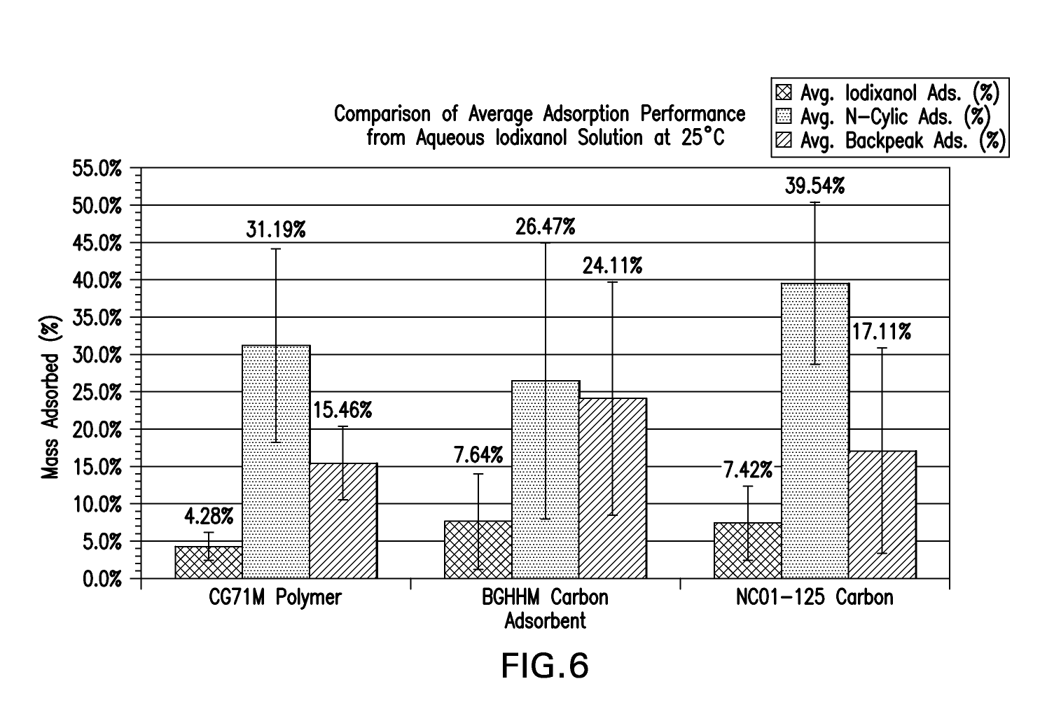

To examine the isotherm data, the average performance of each adsorbent may be plotted. Because the overall shape of the isotherms over these concentration ranges is fairly flat, the average will provide some relative performance of the adsorbent at purifying iodixanol. Such a plot is shown in FIG. 6. The performance is based on the percentage of the original mass in the solution that is adsorbed by the adsorbent. The standard deviations are also given, and are large due to the nature of the average measurement being done over a fairly wide concentration range. On average, all three adsorbents remove about 15-25% of the initial backpeak level (low concentration), while adsorbing only 4-7% of the iodixanol (high concentration). This indicates that the adsorbents are highly selective for the backpeaks relative to the iodixanol.

From the measurements, an average separation factor can also be calculated. The separation factor is defined as the ratio of the mole fractions of components i and j in phases 1 and 2. See King, C. J. Separation Processes. 1980: McGraw-Hill.

$${α}_{ij} = \frac{{x}_{i 1} / {x}_{j 1}}{{x}_{i 2} / {x}_{j 2}} \qquad Eq . 1$$

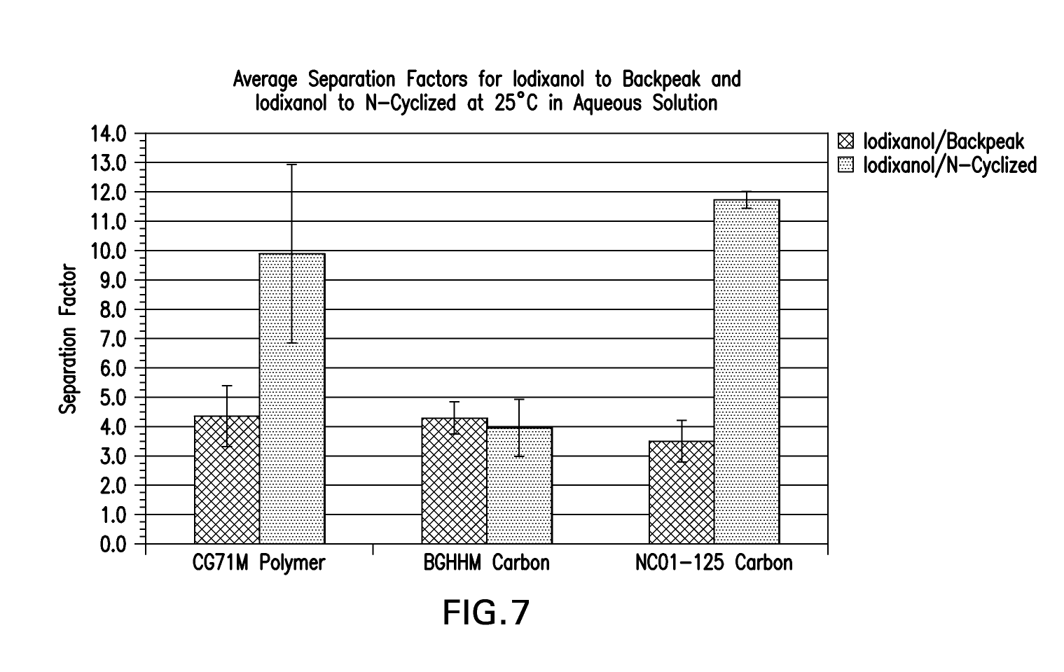

For aij>1, component i will be concentrated in phase 1, and component j in phase 2. In this case, iodixanol is component i and backpeak or N-cyclized is component j, with phase 1 being the solution and phase 2 being the solid adsorbent. As can be seen in FIG. 7, the average separation factors for iodixanol to backpeaks and iodixanol to N-acetyl cyclic iodixanol are shown. All are greater than one, indicating that iodixanol is concentrated in the solution phase, and the backpeaks and N-acetyl cyclic iodixanol are concentrated in the adsorbed phase. The larger the positive number, the better the separation. The N-acetyl cyclic iodixanol is less polar and thus separated better from the iodixanol than the backpeaks. All three non-polar adsorbents are about equal at backpeak separation, with the CG71M and the NC01-125 being better at N-acetyl cyclic iodixanol separation. This separation factor comparison may be a preferred method of comparing adsorption performance over the range of concentrations than the isotherms, due to the potential inaccuracies in calculations noted above.

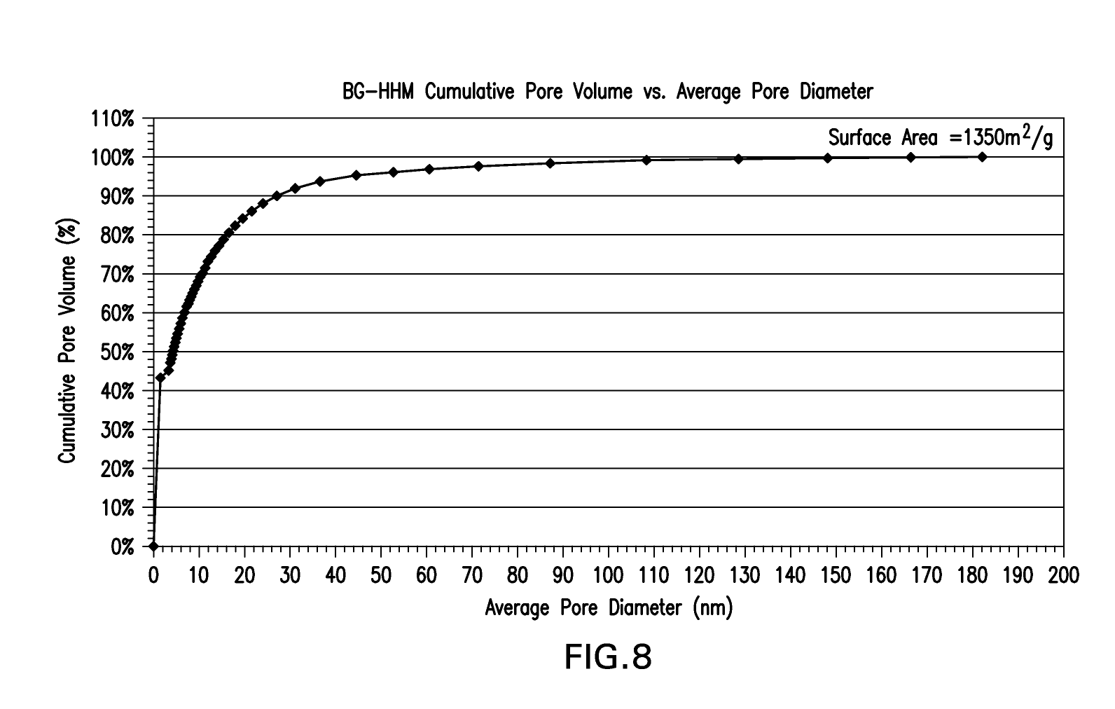

A study of Calgon BGHHM carbon pore size distribution (see FIG. 8) was performed, and is shown to give an example of the pore sizes that contribute to the adsorptive purification of iodixanol. In this way, the preferred pore sizes for purification may be determined in order to optimize adsorbent performance.

From the cumulative pore volume for BGHHM carbon, it can be seen that 90% of the pore volume is less than 30 nm, making the adsorbent highly micro-porous. The surface area for adsorption is about 1350 m²/g. For comparison, the CG71M acrylic ester resin has an average pore diameter of 25 nm and a surface area of 500 m²/g, according to the manufacturer. Further characterization of all the promising adsorbents should be performed, to confirm that micro-porous adsorbents with a majority of the non-polar pores less than 30 nm are preferred adsorbents for separating the impurities from the iodixanol in polar solvents.

The invention is illustrated further by the following examples that are not to be construed as limiting the invention in scope to the specific procedures or products described in them.

### EXAMPLES

### Example 1

### Reaction Conditions

Reagents for adsorbent studies: 2-Methoxyethanol (2-ME; Aldrich, HPLC grade, >=99.9%, Lot#03758HE) was used without further purification. De-ionized water was provided by a Millipore Synergy 185 system, giving 18 mega-ohm water. Iodixanol solids were from Lindesnes Batch #10429846. Adsorbents used and their manufacturers are listed in Table 1.

TABLE 1
Adsorbent types and manufacturers used for iodixanol purification

| | |
|---|---|
| Adsorbent | Manufacturer |

Activated Carbons

| | |
|---|---|
| GC 8x30S | General Carbon |
| Carbsorb 40 | Calgon |
| CPG | Calgon |
| OLC | Calgon |
| BL | Calgon |
| BG-HHM | Calgon |
| NC01-125 | Norit |
| M-1311 | Norit |

Polymers

| | |
|---|---|
| CG161M Acrylic Ester | Rohm & Haas |
| CG71M Acrylic Ester | Rohm & Haas |
| XAD-7 Acrylic Ester | Rohm & Haas |
| XAD-4 Di-Vinyl Benzene | Rohm & Haas |
| XSD-2 Di-Vinyl Benzene | Rohm & Haas |

Silicas

| | |
|---|---|
| C930 | PQ Corporation |
| D350EL | PQ Corporation |

In addition, some other adsorbents were identified using the Supelco catalog, as potentially effective for these types of separations, and are listed in Table 2.

TABLE 2
Listing of various polar and non-polar adsorbents available from Supelco

| | | | | | Surface A | Pore V | Mean pore | | |
|---|---|---|---|---|---|---|---|---|---|
| Adsorbent | Supelco # | Manufacturer | CAS # | Composition | (m²/g) | (ml/g) | size (nm) | Particle Size | |
| Supelite DAX-8 | 20278 | Supelco | | Acrylic Ester | 160 | 0.79 | 22.5 | 40-60 | Mesh |
| Combigel (Amberlite) | 502537B | Supelco, R&H | | Underivatized | | | | 50-100 | Mesh |
| XE-305 | | | | polystyrene | | | | | |
| P4EDVB | 42696-2 | Supelco | 9043-77-0 | Poly(4-ethylsyrene-co- | | | 10.0 | 300-1200 | μm |
| | | | | DVB) | | | | | |
| P4EDVB | 42696-2 | Supelco | 9043-77-0 | Poly(4-ethylsyrene-co- | | | 7.5 | 500 | μm |
| | | | | DVB) | | | | | |
| PS-DVB | 42698-9 | Supelco | 9003-70-7 | Poly(styrene-co-DVB) | | | 4.6 | 300-800 | μm |
| Amberlite XAD7HP | 13361-U | R&H | 37380-43-1 | Acrylic Ester | 450 | 1.14 | 9.0 | 20-60 | Mesh |
| Amberlite XAD16HP | 13355-U | R&H | 9003-69-4 | PS-DVB | 800 | 1.82 | 10.0 | 20-60 | Mesh |
| Amberlite XAD1180 | 10377 | R&H | 97396-56-0 | | 500 | | | | |
| Amberlite XAD761 | 10356 | R&H | | phenol- | 300 | 0.43 | 60.0 | 16-50 | Mesh |
| | | | | formaldehyde/methylol | | | | | |
| | | | | FG | | | | | |
| Amberchrom CG761 | 10366 | R&H | | Acrylic ester | 500 | 1.17 | 25.0 | 80-160 | μm |
| Amberchrom CG161 | 10369 | R&H | | PS-DVB | 900 | 1.45 | 15.0 | 50-100 | μm |
| Amberchrom CG300 | 13908 | R&H | | PS-DVB | 700 | 1.66 | 30.0 | 25-50 | μm |
| Amberchrom CG1000S | 13911 | R&H | | PS-DVB | 250 | 1.66 | 100.0 | 25-50 | μm |
| Diaion HP-20 | 13605 | Mitsubishi | | PS-DVB | 500 | 1.3 | 26.0 | 250-850 | μm |
| Diaion HP-20SS | 13613-U | Mitsubishi | | PS-DVB | 500 | 1.3 | 26.0 | 75-150 | μm |
| Diaion HP-2MG | 13601 | Mitsubishi | | PMA | 500 | 1.2 | 17.0 | 25-50 | μm |
| Sepabeads SP-20SS | 13617-U | Mitsubishi | | PS-DVB | 500 | 1.01 | 26.0 | 50-100 | μm |

Adsorption Experimental: Adsorption studies used a standard solution of iodixanol in 2-ME or de-ionized water. Several controls of each solution were measured for iodixanol, backpeak and N-acetyl cyclics via HPLC. HPLC samples of solutions used in controls and adsorption experiments were prepared from a weighed 125-0 aliquot diluted in a weighed amount of 25 ml de-ionized water.

Silica and carbon adsorbents were prepared by drying at 250° C. and 200 mm Hg vacuum in an oven in order to remove any adsorbed species prior to mixing with the solution. Polymer adsorbents were dried at 100° C. and 200 mm Hg.

The adsorption was performed by weighing out 10 ml of standard solution into a 50-ml jar. Next a weighed amount of adsorbent was added to the jar, along with a Teflon stir bar. The jar was then sealed under nitrogen and mixed on a stir plate for 24-hours. The temperature of the ambient air was also monitored, and fluctuated between 22-27° C.

After equilibration, a portion of the resulting solution was filtered using a 0.5 μm Teflon syringe filter to remove any adsorbent particles. Then a 125-μl aliquot of this filtered solution was diluted in a weighed amount of 25 ml de-ionized water. This diluted sample was then analyzed via HPLC.

Subsequently, the areas for peaks of the known standard solutions were used to calculate the amount of iodixanol, backpeak and N-acetyl cyclics in the equilibrated adsorption solution. The dilution factor was taken into account, and the amounts of each component in the solution and adsorbed phases were calculated.

HPLC Analysis of Adsorbent Solutions: HPLC analysis was performed on an Agilent 1100 series HPLC as indicated below.
Column: YMC-Pack ODS-AM, S-5 μm, 120A, 15 cm and diameter 4.6 mm,
Mobile phase:—Millipore Synergy 185 system de-ionized water.
—50% Acetonitrile in Millipore Synergy 185 system de-ionized water.
Flow: 1.25 ml/min.
Detector: UV—254 nm (15 μl flow cell and 10 mm path length)
Temperature: 25° C. or ambient

### Gradient:

| | 50% ACN in | |
|---|---|---|
| | de-ionized | De-ionized |
| Time (min) | water | water |
| 0.0 | 6 | 94 |
| 2.7 | 6 | 94 |
| 5.5 | 14 | 86 |
| 16.5 | 14 | 86 |
| 19.5 | 26 | 74 |
| 26.5 | 90 | 10 |
| 31.5 | 90 | 10 |

### Example 2

### Silica Adsorption Using 2-Methoxyethanol Solution

157.5 g of iodixanol (GE Healthcare Lot#1042986) was dissolved in 771.7 g of 2-Methoxyethanol (Aldrich Lot#03758HE) to give a 16.95 Wt % solids (iodixanol and impurities) in solution. 25 ml aliquots of this solution were added to a glass jar with a magnetic stir bar. All adsorbents were dried at 250° C. and 250 mm Hg vacuum in a drying oven. Then a weighed amount of dry adsorbent was also added to the jars, except for the control, where no adsorbent was added. The amounts are summarized in Table 3, to give various iodixanol to adsorbent ratios. The glass jars were then placed on a magnetic stirring board and allowed to stir and equilibrate for 20 hours. The subsequent mother liquor was then filtered using a 2-micron Teflon syringe filter. A 125-μl aliquot of this filtered solution was then diluted in 25 ml of 16 mega-ohm purified water and analyzed for the amount of iodixanol, backpeaks and N-acetyl cyclized solids remaining in the solution. The relative amount of each component in each solution was then compared to the control to determine how effective the adsorbent was at removing the impurities. The data in Table 3 shows that the silica adsorbents are not effective at separating out the non-polar impurities from the iodixanol in 2-Methoxyethanol.

TABLE 3
Summary of Silica Adsorption Experiments using Iodixanol dissolved in 2-Methoxyethanol

| | Notebook # | | | | |
|---|---|---|---|---|---|
| | AS326-78-1 | AS326-78-2 | AS326-78-3 | AS326-78-4 | AS326-78-8 |
| Solution Mass (g) | 26.43 | 26.48 | 26.80 | 26.47 | 26.51 |
| Iodixanol Content (g) | 4.4799 | 4.4884 | 4.5426 | 4.4867 | 4.4934 |
| Adsorbent Type | None | D350EL Silica | D350EL Silica | D350EL Silica | R100 Silica |
| Adsorbent Mass (g) | — | 0.44 | 0.91 | 2.24 | 2.25 |
| Manufacturer | — | PQ Corp. | PQ Corp. | PQ Corp. | PQ Corp. |
| Lot # | — | S009 | S009 | S009 | R40507313 |
| Iodixanol Mass/Solids Mass | — | 10.20 | 4.99 | 2.00 | 2.00 |
| Drying T (C.) | — | 250 | 250 | 250 | 250 |
| Equilibrium Analysis | | | | | |
| (Normalized % component in | | | | | |
| solution) | | | | | |
| Iodixanol | 99.1918 | 99.0714 | 99.1934 | 99.0780 | 99.2387 |
| Backpeaks | 0.7548 | 0.8144 | 0.7349 | 0.8084 | 0.7060 |
| N-acetyl-cyclized | 0.0535 | 0.1142 | 0.0717 | 0.1136 | 0.0552 |
| | Notebook # | | | | |
| | AS326-78-9 | AS326-78-10 | AS326-78-11 | AS326-78-12 | AS326-78-13 |
| Solution Mass (g) | 26.48 | 26.40 | 26.47 | 26.42 | 26.45 |
| Iodixanol Content (g) | 4.4884 | 4.4850 | 4.4867 | 4.4782 | 4.4833 |
| Adsorbent Type | R100 Silica | R100 Silica | C930 Silica | C930 Silica | C930 Silica |
| Adsorbent Mass (g) | 0.89 | 0.44 | 0.44 | 0.89 | 2.24 |
| Manufacturer | PQ Corp. | PQ Corp. | PQ Corp. | PQ Corp. | PQ Corp. |
| Lot # | R40507313 | R40507313 | 52642 | 52642 | 52642 |
| Iodixanol Mass/Solids Mass | 5.04 | 10.19 | 10.20 | 5.03 | 2.00 |
| Drying T (C.) | 250 | 250 | 250 | 250 | 250 |
| Equilibrium Analysis | | | | | |
| (Normalized % component in | | | | | |
| solution) | | | | | |
| Iodixanol | 98.9785 | 99.0692 | 98.9975 | 99.0964 | 99.1139 |
| Backpeaks | 0.9206 | 0.8258 | 0.8959 | 0.8168 | 0.8106 |
| N-acetyl-cyclized | 0.1010 | 0.1050 | 0.1066 | 0.0869 | 0.0755 |

### Example 3

### Alumina Adsorption Using 2-Methoxyethanol Solution

Using the same control solution prepared in Example 2, and the same method of drying the adsorbents, a similar set of experiments was done using Alumina adsorbent. The amounts are summarized in Table 4, to give various iodixanol to adsorbent ratios. The glass jars were then placed on a magnetic stirring board and allowed to stir and equilibrate for 20 hours. The subsequent mother liquor was then filtered using a 2-micron Teflon syringe filter. A 125-μl aliquot of this filtered solution was then diluted in 25 ml of 16 mega-ohm purified water and analyzed for the amount of iodixanol, backpeaks and N-acetyl cyclized solids remaining in the solution. The relative amount of each component in each solution was then compared to the control to determine how effective the adsorbent was at removing the impurities. The data in Table 4 show that the alumina adsorbents are not effective at separating out the non-polar impurities from the iodixanol in 2-Methoxyethanol, and in fact due to the acidic nature of the adsorbent surface, they catalyze the reaction of iodixanol to the N-acetyl cyclized impurity. The higher the amount of adsorbent added, the larger the N-acetyl cyclized impurity formed.

TABLE 4
Summary of Alumina Adsorption Experiments using iodixanol dissolved in 2-Methoxyethanol

| | Notebook # | | | |
|---|---|---|---|---|
| | AS326-78-1 | AS326-78-5 | AS326-78-6 | AS326-78-7 |
| Solution Mass (g) | 26.43 | 26.50 | 26.44 | 26.50 |
| Iodixanol Content (g) | 4.4799 | 4.4918 | 4.4816 | 4.4918 |
| Adsorbent Type | None | Alumina 9139A | Alumina 9139A | Alumina 9139A |
| Adsorbent Mass (g) | — | 0.46 | 0.89 | 2.25 |
| Manufacturer | — | UOP | UOP | UOP |
| Lot # | — | 4413000226 | 4413000226 | 4413000226 |
| Iodixanol Mass/Solids Mass | — | 9.76 | 5.04 | 2.00 |
| Drying T (C.) | — | 250 | 250 | 250 |
| Equilibrium Analysis | | | | |
| (Normalized % | | | | |
| component in | | | | |
| solution) | | | | |
| Iodixanol | 99.1918 | 98.1269 | 97.5749 | 96.6652 |
| Backpeaks | 0.7548 | 0.7008 | 0.7425 | 0.6906 |
| N-acetyl-cyclized | 0.0535 | 1.1723 | 1.6825 | 2.6443 |

### Example 4

### Polymer Resin Adsorption Using 2-Methoxyethanol Solution

Using the same control solution prepared in Example 2, and the same method of drying the adsorbents at 100° C. and 250 mm Hg vacuum, a similar set of experiments was done using polymeric resin adsorbents. The amounts are summarized in Table 5, to give various iodixanol to adsorbent ratios. The glass jars were then placed on a magnetic stirring board and allowed to stir and equilibrate for 20 hours. The subsequent mother liquor was then filtered using a 2-micron Teflon syringe filter. A 125-μl aliquot of this filtered solution was then diluted in 25 ml of 16 mega-ohm purified water and analyzed for the amount of iodixanol, backpeaks and N-acetyl cyclized solids remaining in the solution. The relative amount of each component in each solution was then compared to the control to determine how effective the adsorbent was at removing the impurities. The data in Table 5 shows that the polymer adsorbents are not effective at separating out the non-polar impurities from the iodixanol in 2-Methoxyethanol. Again, due to the acidic nature of the adsorbent surface, some polymer resins catalyze the reaction of iodixanol to the N-acetyl cyclized impurity.

TABLE 5
Summary of Resin Adsorption Experiments Using Iodixanol Dissolved in 2-Methoxyethanol

| | Notebook # | | | |
|---|---|---|---|---|
| | AS326-78-1 | AS326-79-18 | AS326-79-19 | AS326-79-20 |
| Solution Mass (g) | 26.43 | 26.54 | 26.47 | 26.43 |
| Iodixanol Content (g) | 4.4799 | 4.4980 | 4.4862 | 4.4804 |
| Adsorbent Type | None | Amberlite XAD-2 | Amberlite XAD-2 | Amberlite XAD-4 |
| Adsorbent Mass (g) | — | 2.005 | 0.408 | 2.003 |
| Manufacturer | — | Rohm & Haas | Rohm & Haas | Rohm & Haas |
| Lot # | — | 04917TZ | 04917TZ | 03803HF |
| Iodixanol Mass/Solids Mass | — | 2.24 | 11.00 | 2.24 |
| Drying T (C.) | — | 100 | 100 | 100 |
| Equilibrium Analysis | | | | |
| (Normalized % component in | | | | |
| solution) | | | | |
| Iodixanol | 99.1918 | 98.2166 | 98.9811 | 98.5871 |
| Backpeaks | 0.7548 | 0.7480 | 0.7729 | 0.7597 |
| N-acetyl-cyclized | 0.0535 | 1.0354 | 0.2460 | 0.6532 |

| | Notebook # | | | |
|---|---|---|---|---|
| | AS326-79-21 | AS326-79-22 | AS326-79-23 | AS326-79-24 |
| Solution Mass (g) | 26.36 | 26.40 | 26.45 | 26.50 |
| Iodixanol Content (g) | 4.4675 | 4.4751 | 4.4824 | 4.4923 |
| Adsorbent Type | Amberlite XAD-7 | Dowex MSA | IRC76 | Amberlyst 15 |
| Adsorbent Mass (g) | 2.002 | 2.003 | 2.003 | 2.003 |
| Manufacturer | Rohm & Haas | Dow | Rohm & Haas | Rohm & Haas |
| Lot # | 03729DF | 13107EA | 6.22E+06 | 210126 |
| Iodixanol Mass/Solids Mass | 2.23 | 2.23 | 2.24 | 2.24 |
| Drying T (C.) | 100 | 100 | 100 | 100 |
| Equilibrium Analysis | | | | |
| (Normalized % component in | | | | |
| solution) | | | | |
| Iodixanol | 99.2723 | 99.1813 | 99.1963 | 99.0813 |
| Backpeaks | 0.6807 | 0.7596 | 0.7389 | 0.8711 |
| N-acetyl-cyclized | 0.0470 | 0.0590 | 0.0648 | 0.0476 |

### Example 5

### Further Polymer Resin Adsorption Using 2-Methoxyethanol Solution

Using the same control solution prepared in Example 2, and the same method of drying the adsorbents at 40° C. and 250 mm Hg vacuum, a similar set of experiments to Example 4 was done using new set of non-polar polymeric resin adsorbents. The amounts are summarized in Table 6, to give various iodixanol to adsorbent ratios. The glass jars were then placed on a magnetic stirring board and allowed to stir and equilibrate for 20 hours. The subsequent mother liquor was then filtered using a 2-micron Teflon syringe filter. A 125-μl aliquot of this filtered solution was then diluted in 25 ml of 16 mega-ohm purified water and analyzed for the amount of iodixanol, backpeaks and N-acetyl cyclized solids remaining in the solution. The relative amount of each component in each solution was then compared to the control to determine how effective the adsorbent was at removing the impurities. The data in Table 6 shows that the polymer adsorbents are not effective at separating out the non-polar impurities from the iodixanol in 2-Methoxyethanol.

TABLE 6
Summary of Resin Adsorption Experiments using Iodixanol dissolved in 2-Methoxyethanol

| | Notebook # | | | | | |
|---|---|---|---|---|---|---|
| | AS326-99-1 | AS326-99-2 | AS326-99-3 | AS326-99-4 | AS326-99-5 | AS326-99-6 |
| Solution Mass (g) | 6.9212 | 6.7906 | 7.1947 | 7.1462 | 7.1619 | 7.1891 |
| Iodixanol Content (g) | 1.1731 | 1.1510 | 1.2195 | 1.2113 | 1.2139 | 1.2186 |
| Adsorbent Type | None | CG71M | CG71M | None | CG161M | CG161M |
| Adsorbent Mass (g) | — | 0.2302 | 0.6114 | — | 0.2428 | 0.6085 |
| Manufacturer | — | Rohm & Haas | Rohm & Haas | — | Rohm & Haas | Rohm & Haas |
| Lot # | — | 0003604478 | 0003604478 | — | 0003558506 | 0003558506 |
| Iodixanol Mass/Solids Mass | — | 5.000 | 1.995 | — | 5.000 | 2.003 |
| Drying T (C.) | — | 100 | 100 | — | 100 | 100 |
| Equilibrium Analysis | | | | | | |
| (Normalized % component in | | | | | | |
| solution) | | | | | | |
| Iodixanol | 99.1302 | 99.0317 | 99.0926 | 99.1604 | 99.2252 | 99.2095 |
| Backpeaks | 0.7859 | 0.8773 | 0.8397 | 0.7760 | 0.7153 | 0.7471 |
| N-acetyl-cyclized | 0.0838 | 0.0910 | 0.0677 | 0.0636 | 0.0595 | 0.0435 |

### Example 6

### Carbon Adsorption Using Aqueous Solution

55.05 g of iodixanol (GE Healthcare Lot#1042986) was dissolved in 220.1 g of 16 mega-ohm purified water to give a 20.76 Wt % solids (iodixanol and impurities) in solution. 10 ml aliquots of this solution were added to a glass jar with a magnetic stir bar. All adsorbents were dried at 250° C. and 250 mm Hg vacuum in a drying oven. Then a weighed amount of dry adsorbent was also added to the jars, except for the control, where no adsorbent was added. The amounts are summarized in Table 7, to give various iodixanol to adsorbent ratios. The glass jars were then placed on a magnetic stirring board and allowed to stir and equilibrate for 20 hours. The subsequent mother liquor was then filtered using a 2-micron Teflon syringe filter. A 125-μl aliquot of this filtered solution was then diluted in 25 ml of 16 mega-ohm purified water and analyzed for the amount of iodixanol, backpeaks and N-acetyl cyclized solids remaining in the solution. The relative amount of each component in each solution was then compared to the control to determine how effective the adsorbent was at removing the impurities. The data in Table 7 shows that some carbon adsorbents with the right properties are effective at separating out the non-polar impurities from the iodixanol in a polar water solution.

TABLE 7
Summary of Carbon Adsorption Experiments using Iodixanol dissolved in water

| | Notebook # | | | | |
|---|---|---|---|---|---|
| | AS99-7 | AS99-8 | AS99-9 | AS99-10 | AS99-11 |
| Solution Mass (g) | 10.9606 | 10.9628 | 10.9130 | 11.0316 | 10.9681 |
| Iodixanol Content (g) | 2.2754 | 2.2759 | 2.2655 | 2.2902 | 2.2770 |
| Adsorbent Type | None | Carbon 8x30S | Carbsorb 40 | CPG Carbon | OLC Carbon |
| Adsorbent Mass (g) | — | 1.1387 | 1.1329 | 1.1452 | 1.1383 |
| Manufacturer | — | General Carbon | Calgon | Calgon | Calagon |
| Lot # | — | None - research | None - research | None - research | None - research |
| Iodixanol Mass/Solids Mass | — | 1.9989 | 1.9997 | 1.9999 | 2.0004 |
| Drying T (C.) | — | 250 | 250 | 250 | 250 |
| Equilibrium Analysis | | | | | |
| (Normalized % component in | | | | | |
| solution) | | | | | |
| Iodixanol | 99.2216 | 99.2257 | 99.3391 | 99.3555 | 99.2463 |
| Backpeaks | 0.6662 | 0.7398 | 0.5853 | 0.6054 | 0.6835 |
| N-acetyl-cyclized | 0.1122 | 0.0345 | 0.0756 | 0.0391 | 0.0702 |

| | Notebook # | | | |
|---|---|---|---|---|
| | AS99-12 | AS99-13 | AS99-14 | AS99-15 |
| Solution Mass (g) | 11.0744 | 11.0208 | 10.9792 | 11.0233 |
| Iodixanol Content (g) | 2.2990 | 2.2879 | 2.2793 | 2.2884 |
| Adsorbent Type | BL Carbon | BG-HHM Carbon | NC01-125 Carbon | M-1311 Carbon |
| Adsorbent Mass (g) | 1.1518 | 1.1445 | 1.1402 | 1.1449 |
| Manufacturer | Calgon | Calgon | Norit | Norit |
| Lot # | None - research | None - research | None - research | None - research |
| Iodixanol Mass/Solids Mass | 1.9960 | 1.9990 | 1.9990 | 1.9988 |
| Drying T (C.) | 250 | 250 | 250 | 250 |
| Equilibrium Analysis | | | | |
| (Normalized % component in | | | | |
| solution) | | | | |
| Iodixanol | 99.4259 | 99.5267 | 99.5174 | 99.5022 |
| Backpeaks | 0.5547 | 0.4382 | 0.4479 | 0.4704 |
| N-acetyl-cyclized | 0.0194 | 0.0351 | 0.0347 | 0.0275 |

### Example 7

### Polymer Resin and Polar Silica Adsorption Using Aqueous Solution

Using the same control solution prepared in Example 6, and the same method of drying the adsorbents at 100° C. for polymer (40° C. for CG series polymers) and 250° C. for silica and 250 mm Hg vacuum, a similar set of experiments to Example 6 was done using new set of non-polar polymeric resin adsorbents and a set of polar silica for contrast. The amounts are summarized in Table 8, to give various iodixanol to adsorbent ratios. The glass jars were then placed on a magnetic stirring board and allowed to stir and equilibrate for 20 hours. The subsequent mother liquor was then filtered using a 2-micron Teflon syringe filter. A 125-μl aliquot of this filtered solution was then diluted in 25 ml of 16 mega-ohm purified water and analyzed for the amount of iodixanol, backpeaks and N-acetyl cyclized solids remaining in the solution. The relative amount of each component in each solution was then compared to the control to determine how effective the adsorbent was at removing the impurities. The data in Table 8 shows that some polymer adsorbents are effective at separating out the non-polar impurities from the iodixanol in water, while the polar silicas are not effective at iodixanol purification in a polar solvent like water.

TABLE 8
Summary of Resin & Silica Adsorption Experiments using Iodixanol dissolved in water

| | Notebook # | | | | | | | |
|---|---|---|---|---|---|---|---|---|
| | AS99-23 | AS99-16 | AS99-17 | AS99-18 | AS99-19 | AS99-20 | AS99-21 | AS99-22 |
| Solution Mass (g) | 11.1108 | 10.9882 | 10.9293 | 11.0162 | 10.9395 | 10.9812 | 10.8965 | 10.9858 |
| Iodixanol Content (g) | 2.3066 | 2.2812 | 2.2689 | 2.2870 | 2.2710 | 2.2797 | 2.2621 | 2.2807 |
| Adsorbent Type | None | CG161M | CG71M | XAD-7 | C930 Silica | D350EL Silica | XAD-2 | XAD-4 |
| Adsorbent Mass (g) | — | 1.1438 | 1.1345 | 1.1511 | 1.1385 | 1.1422 | 1.1311 | 1.1402 |
| Manufacturer | — | Rohm & | Rohm & | Rohm & | PQ Corp. | PQ Corp. | Rohm & | Rohm & |
| | | Haas | Haas | Haas | | | Haas | Haas |
| Lot # | — | 3558506 | 3604478 | 03729DF | 52642 | S009 | 04917TZ | 03803HF |
| Iodixanol Mass/Solids Mass | — | 1.9944 | 2.0000 | 1.9868 | 1.9947 | 1.9959 | 2.0000 | 2.0003 |
| Drying T (C.) | — | 40 | 40 | 100 | 250 | 250 | 100 | 100 |
| Equilibrium Analysis | | | | | | | | |
| (Normalized % component in | | | | | | | | |
| solution) | | | | | | | | |
| Iodixanol | 99.2391 | 99.301 | 99.558 | 99.431 | 99.2569 | 99.3377 | 99.2592 | 99.214 |
| Backpeaks | 0.6829 | 0.665 | 0.4197 | 0.5205 | 0.6938 | 0.627 | 0.6535 | 0.7246 |
| N-acetyl-cyclized | 0.078 | 0.034 | 0.0222 | 0.0484 | 0.0493 | 0.0352 | 0.0873 | 0.0614 |

All patents, journal articles, publications and other documents discussed and/or cited above are hereby incorporated by reference.

1. A process for purifying bulk iodixanol comprising the step of contacting a solution of bulk iodixanol comprising iodixanol, N-acetyl cyclized iodixanol, cyclized iodixanol, and primary o-alkyl trimer, with non-polar organic adsorbents, where the average pore diameter is smaller than about 30 nm.
